(19) United States
(12) Patent Application Publication
(10) Pub. No.: US 2004/0068180 A1
(43) Pub. Date: Apr. 8, 2004

(54) Rotary ultrasound scanner for soft tissue examination

(76) Inventors: Jeffrey Collins, Belfountain (CA); Bogdan Motoc, Toronto (CA); Karen Saghatelyan, Toronto (CA)

Correspondence Address: Orange & Charl, Suite 4900, 66 Wellington Street West, P.O. Box 190, Toronto, M5K 1H6, CA

(21) Appl. No.: 10/263,758

(22) Filed: Oct. 4, 2002

(51) Int. Cl. A61B 5/05

(52) U.S. Cl. 600/425

(57) ABSTRACT

A medical imaging system comprises a patient support surface. An imaging apparatus has a support table located within the support surface, and adjustable relative to the support surface to be located above the surface and thereby engage a portion of the patient to be imaged. The imaging is performed by a rotary scanner rotatable about a vertical axis utilises multiple transducers that each image a different portion to produce conical slice. The signal processing compensates for attenuation and different gains in the transducers to provide a normalised set f data for reconstruction.

### BACKGROUND OF THE INVENTION

1. Field of the Invention

The present invention relates in general to medical imaging systems and to a rotary ultrasound scanner for soft tissue examination for use in such systems.

2. Description of the Prior Art

Medical imaging is an important aspect in the practice of medicine. The use of medical imaging to assist in the diagnosis of patients has been well established over the years with different technologies being employed in scanning equipment. Current imaging technologies utilise x-rays, magnetic resonance imaging (MRI), ultrasound and other modalities to produce an image of the region of interest. However, each of these suffers from certain limitations in use. Technologies using x-rays require a limited use in a carefully controlled environment in view of the cumulative effect of radiation on the patient. Both MRI and x-ray imaging equipment are relatively immobile due to the large size of the scanning equipment. It is also frequently necessary to inject dyes (contrast media) into the patient to enhance visualisation, which, in turn, leads to the use of further specialized equipment.

The use of immobile specialized equipment makes it difficult to monitor patients frequently due to limited access to the equipment and the need to provide examination facilities. This biases the usage against frequent monitoring and evaluation of changes over time, which are particularly beneficial in the early detection of abnormalities.

The use of ultrasound devices in the examination of patients by medical practitioners has been a well-established practice since the 1950s. Ultrasound has proven to be useful in the field of medical imaging for soft tissue examination, particularly in detection of breast cancer in women. However, it has typically been used as an adjunct to mammography due to its limited scanning depth, lack of image resolution and amount of noise present in the acquired data. Historically, ultrasound has been used post-mammography to determine whether masses in the breast are solid (requiring biopsy) or cystic (benign) but has not generally been considered to be useful in detecting masses in breast tissue that are smaller than 1 mm by 1 mm. The factors influencing this limitation are noise in the acquired data, the inability to replicate scans due to tissue deformation, and operator-dependant image interpretation. In practice, mammography has been found useful to detect microcalcifications as small as 0.3 mm. Microcalcifications generally start as small as 0.1 mm and are considered an early indication of a condition requiring ongoing monitoring.

Although there have been attempts to detect microcalcifications using ultrasound technology, it appears that most attempts have not succeeded. A discussion of ultrasound resolution can be found in the doctoral thesis of Sheila McFarland, UBC. In most of the prior art, it is believed that ultrasound is not capable of detecting microcalcifications with any degree of accuracy which has inhibited its utilisation on a broad basis.

However, the use of ultrasound does not require the elaborate safety precautions necessary with other imaging systems and therefore lends itself to frequent monitoring regimes. To be effective, a good coupling must be provided between the ultrasound transducer and the soft tissue and in some prior art scanners, the body part being examined has to be deformed prior to scanning to achieve this. This is partially to secure contact but primarily to manipulate tissues in the insonified area for best identification. Classic ultrasound may be considered a tissue manipulation practice performed with visual assistance. The deformation of the body part makes comparison of sequential images difficult, makes a read difficult to repeat and may lead to various masses being hidden from the scanner.

An ultrasound scanner for imaging breast tissue is disclosed in U.S. Pat. No. 6,005,916 to Johnson et al. and assigned to Techniscan Inc. This scanner utilises an annular array of transducers to interrogate an object placed within a water-filled chamber. The transducers in the annular array are operated sequentially to obtain a circumferential scan and the array is then moved vertically to perform a further scan. In this manner a 3 dimensional image may be obtained. The array may be adjusted circumferentially to adjust the position of the transducers relative to an object.

The scanner disclosed in the Johnson patent is relatively expensive due to its annular array and requires a complicated bladder arrangement for coupling the transducer to the fluid in the examination chamber. Moreover, the Johnson patent is silent as to issues regarding repeatability of image capture such as is necessary to permit monitoring over an extended period.

It is an object of the present invention to mitigate or obviate at least one of the above-mentioned disadvantages.

### SUMMARY OF THE INVENTION

In one aspect, the present invention provides a medical imaging system comprising a patient support surface. An imaging apparatus having a support table located within the support surface, and adjustable relative to the support surface to be located above the surface and thereby engage a portion of the patient to be imaged.

According to a further aspect of the present invention there is provided an ultrasound scanner assembly comprising a base, a drum rotating on the base. A transducer head rotating with the drum and displaced relative to the drum along an axis parallel to the axis of rotation. The head including a plurality of transducers each operable to propagate a wave along an axis of propagation and to receive signals from respective focal zones spaced relative to one another along the axis of propagation.

According also to the present invention there is provided a method of monitoring a medical condition through insonification with ultrasound by locating a portion of a patient on a scanner in a predetermined position to permit acquisition of date in a repeatable manner, conducting a succession of scans of the area of interest of the patient at predetermined intervals, transferring the scan to a remote location and comparing time separated scans to determine changes in said medical condition.

### BRIEF DESCRIPTION OF THE DRAWINGS

An embodiment of the invention will now be described by way of example only with reference to the accompanying drawings in which:

FIG. 1 is a schematic representation of components of a scanning system;

FIG. 2 is a plan view of the system of FIG. 1;

FIG. 3 is a perspective view of a scanner assembly incorporated in the system of FIG. 1;

FIG. 4 is a side elevation of the scanner assembly of FIG. 3;

FIG. 5 is a detailed view of a portion of FIG. 4.

FIG. 6 is a functional block diagram of the controls used in the scanner assembly of FIG. 3;

FIG. 7 is a schematic representation of the data acquired after one pass of the scanner assembly of FIG. 3;

FIG. 8 is a flow chart representing the manipulation of data obtained from a pass of the scanner assembly of FIG. 3;

FIG. 9 is a schematic representation of a complete signal produced by the manipulation of data;

FIG. 10 is a schematic representation of the data structure used in the manipulation process of FIG. 8; and

FIG. 11 is a view of an interface used to control operation of the system of FIG. 1.

### DESCRIPTION OF THE PREFERRED EMBODIMENTS

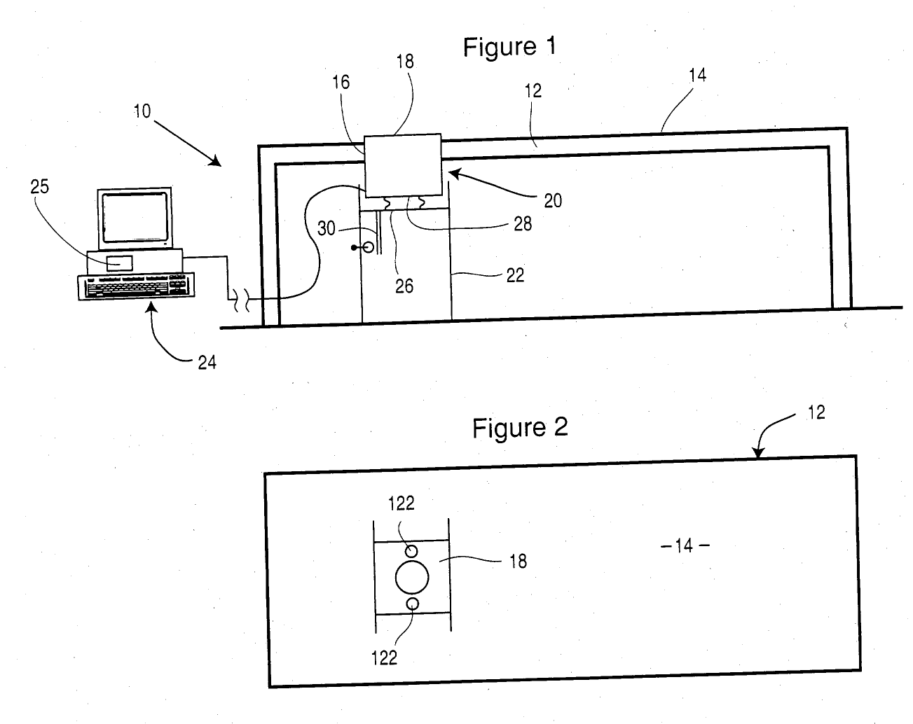

As can be seen in FIGS. 1 and 2, an ultrasound scanning system 10 includes a patient examination station 12 with a patient support surface 14. The surface 14 has an opening 16 to receive a table 18 formed by a lid 19 of a scanner assembly 20. The table 18 may be covered with a soft or cushioning material to improve patient comfort in use if so required. Scanner assembly 20 rests within a scanner case 22 located underneath the examination station 12. A remote computer 24, including a data acquisition software module 25, is connected to the scanner 20 to receive data acquired during the operation of the scanner 20.

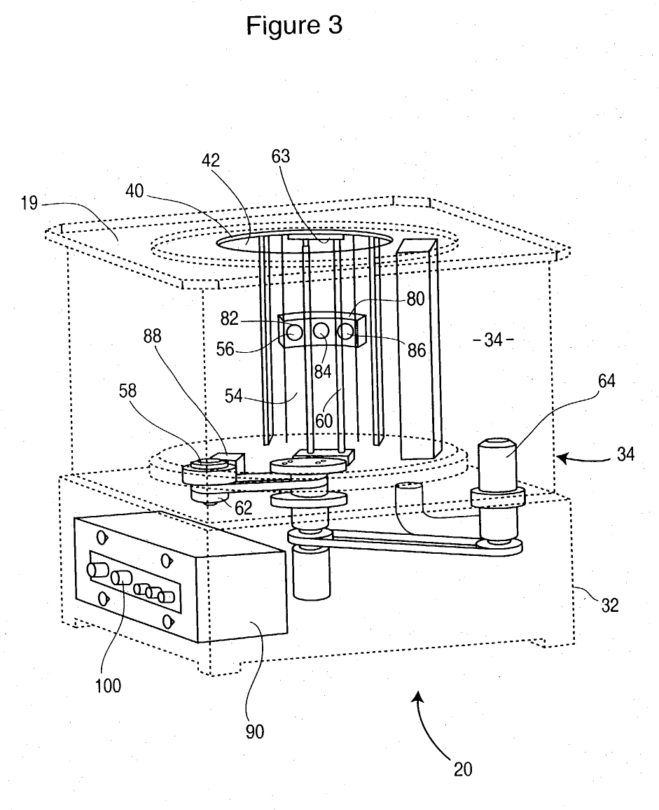

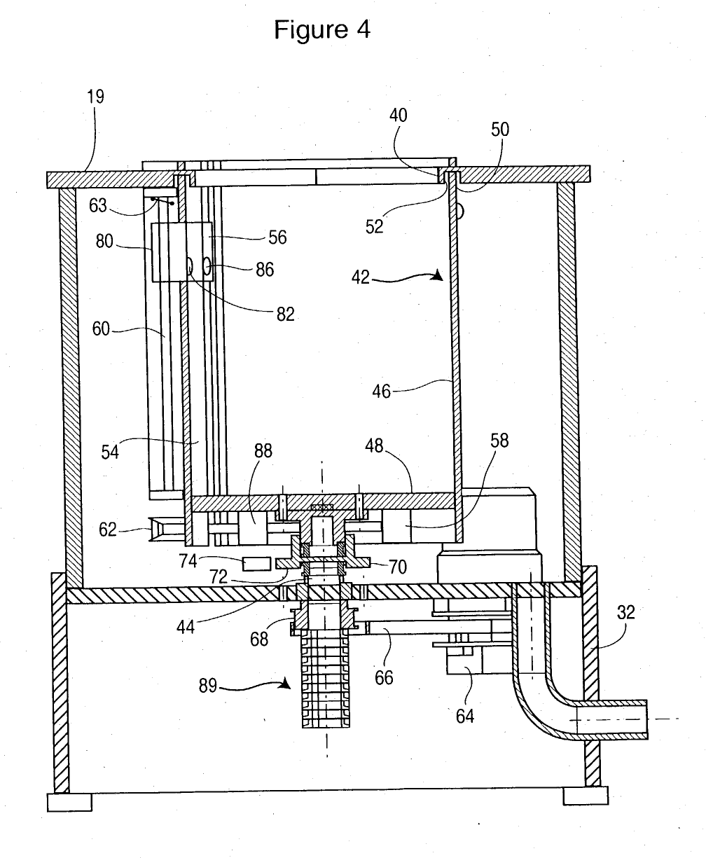

The scanner case 22 is supported on the floor and has an upper support base 26 to receive the scanner assembly 20. A resilient support 28 is positioned between the assembly 20 and support base 26 to allow limited vertical movement between the base 26 and the assembly 20. An adjustment mechanism 30 is provided for the base 26 to allow vertical adjustment of the base and thereby adjust alignment between the patient support surface 14 and the table 18 of the scanner assembly 20. Any suitable adjustment mechanism may be used such as a rack and pinion drive or a releasable cam and slide. The scanner assembly 10 is shown in greater detail in FIGS. 3, 4 and 5.

The scanner assembly 20 includes a base 32 supporting a body 34. Body 34 has sidewalls 36 that support the scanner lid 19 forming the table 18. An aperture 40 is provided in the scanner lid 19 to receive a pendant breast of a patient prone on the support surface 14.

A drum 42 is rotatably supported on the base 32 by a spindle 44. The drum 42 has a peripheral sidewall 46 and an end plate 48 supported on the spindle 44. The upper end 50 of the sidewall is received in a recess 52 in the underside of the lid 19 around the aperture 40 to locate the drum but allow rotation relative to the scanner lid 19.

A channel 54 is formed in the sidewall 46 parallel to axis of rotation. The channel 54 supports a transducer head 56 that is moveable along the channel 54 under the control of a drive motor 58. The motor 58 is supported on the underside of the end plate 48 and drives a pair of lead screws 60 through a toothed belt 62. The lead screws 60 support the transducer head 56 so that rotation of the lead screws will provide translation of the transducer head 56 along the channel 54. A switch 63 is located at an upper end of the channel 54 and is activated by the transducer head 56 to indicate an upper limit of travel.

The spindle 44 supporting the drum 42 is driven by a motor 64 secured to the base and driving the spindle 44 through a toothed belt drive 66 acting on a cog wheel 68. The spindle 44 also carries a rotary encoder 70, which consists of a disc 72 having alternating light and dark sectors disposed around the periphery of the disc. An optical pick-up mechanism 74 is disposed at the periphery of the encoder disc 72 to provide a signal indicative of rotation and position of the drum 42. Each pulse of this signal marks a rotational increment. A resolution of 1800 marks has been found to be satisfactory although other resolutions might be used. The encoder disc 72 also provides a zero home position indicator that allows a reference position for the disc to be determined. The zero position is identifiable in the pulse train obtained from the encoder disc by a unique pattern. Alternatively an absolute position encoder may be used to report a crisp position pointer.

The transducer head 56 includes a substrate 80 (FIG. 3) carrying three ultrasound transducers 82, 84, 86. Each of the transducers is controlled by electronic circuitry indicated at 88 and carried on the end plate 48 of the drum 42. Data flow and control signals to and from the transducers is transferred from the electronic circuitry 80 through slip rings 89 on the spindle 44 below the encoder disk 72 to a buffer and communication module 90 for transfer to the data acquisition module 25 on the computer 24. It will be noted that the location of the circuitry 88 on the rotating components enables the signals from the transducers to be processed without being subjected to the noise that may be introduced by the slip rings 89 and thereby enhance the extraction of information from the resultant signals.

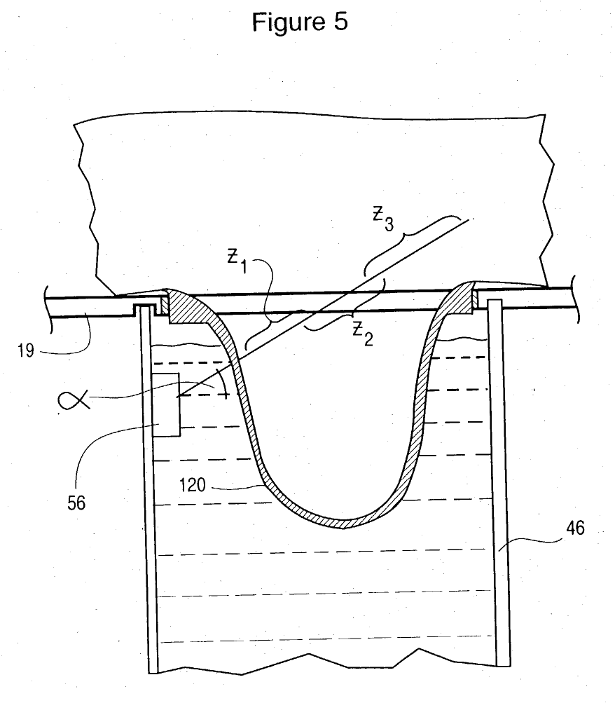

The transducers 82, 84, 86 are each mounted on the substrate 80 such that there axis of propagation is inclined upwardly relative to the axis of rotation of the drum 44. As shown in FIG. 5, the angle of inclination α, measured from the horizontal is selected so that the axis of propagation is generally orthogonal to the average tangent to the surface of a breast located in the drum 44. Typically an angle of 30° to the horizontal (i.e. 60° to the axis of the rotation) is found to be satisfactory although a range of 25° to 35° (55° to 65° to the axis of rotation) may be used.

Each of the transducers is also selected to provide different focal zones Z₁ Z₂ Z₃ which overlap along the axis of propagation. In a preferred embodiment transducers having a focal length of 30, 50 and 80 mm and with depths of field of 22-43 mm; 14-87 mm; and 44-140 mm respectively are used with a drum diameter of 175 mm. The transducers have an operating frequency of 4.0 MHz. A sampling rate of 20 MHz is preferred to provide an oversampling and resolution better than one half wavelength.

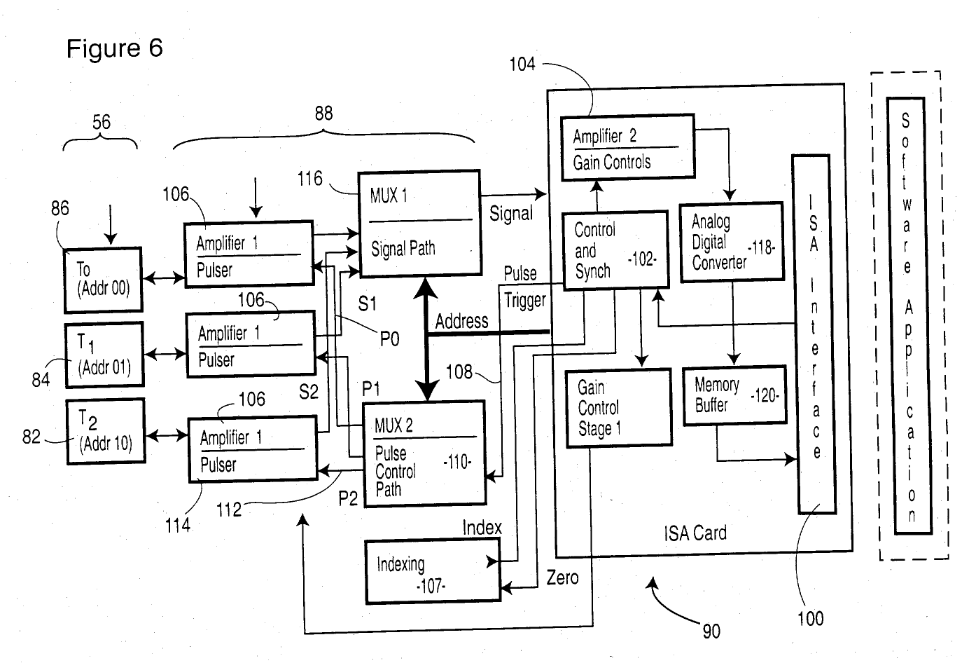

The functional blocks of the electrical circuitry 88 and buffer and communication module 90 are shown in FIG. 6. The buffer and communication module 90 is provided on a standard parallel interface 100 for communication with the computer 24. Overall control of the scanner assembly is provided by a control and synch module 102 that receives operational parameters from the computer 24 as will be described more fully below. The control and synch module 102 initially sets the required gain of a main amplifier 104. Output from the main amplifier 104 is digitised by an A/D converter 118 and stored in a buffer 120 for communication over the interface 100.

A preamplifier 106 is associated with each of the transducers 82, 84, 86 and included in the circuitry 88 carried on the end plate 48. Input signals from the encoder 70 are processed through indexing function 107 and provided to the control and sync module 102. Module 102 generates a trigger pulse 108 at intervals defined by the operational parameters received from the computer 24 and transfers it across the slip ring 89 to a pulse multiplexer control module 110. The multiplexer control module 110 sequentially directs a trigger pulse signal 112 to a pulse circuit 114 associated with each of the transducers 82, 84, 86 respectively. The encoder 70 provides a pulse for each mark on the disc and, for maximum resolution, each pulse is used to trigger one of the pulse circuits 114. Thus for an 1800 line encoder each of the transducers is fired 600 times per revolution.

The reflected signal is received by the transducer and amplified by the corresponding preamplifier 106 before being passed to a signal multiplexer 116. The signal multiplexer 116 is switched by the module 102 after a predefined delay so as to receive the reflected signal in the zone to be examined by the respective transducer. The delay is software adjustable depending on the volume of tissue to be scanned but delays of 30 microseconds (μs) are typical for the exemplified apparatus. Multiplexer 116 passes the composite signal to the main amplifier 104. The amplified signal is then digitised by A/D converter 118 and stored temporarily in buffer 120. The contents of the buffer 120 are read prior to the data being overwritten with the output from the next transducer and are downloaded through interface 100 to the data acquisition module 25 of the computer 24.

The control and synch module 102 is also operable to identify a zero or home position on the encoder 70 and initiate operation of vertical drive motor 58 to displace the transducer head 56 by a selected distance upon completion of a circumferential scan. Thereafter further scanning in a circumferential direction is performed.

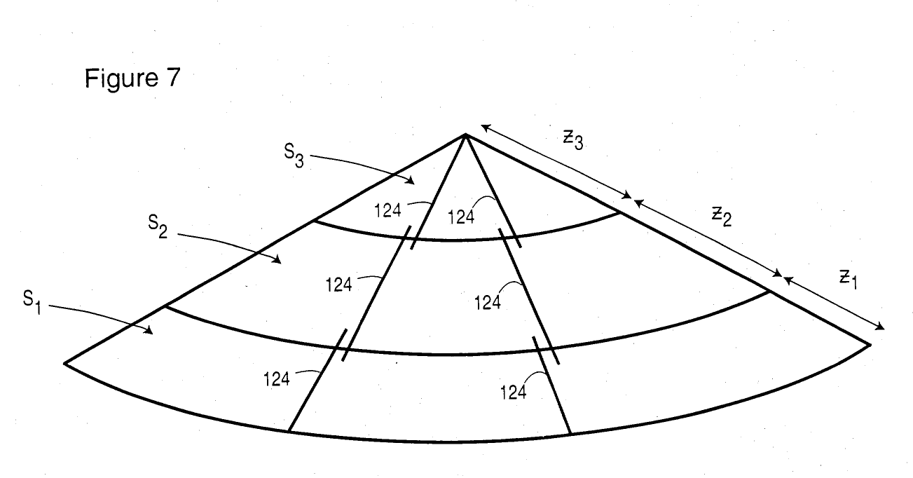

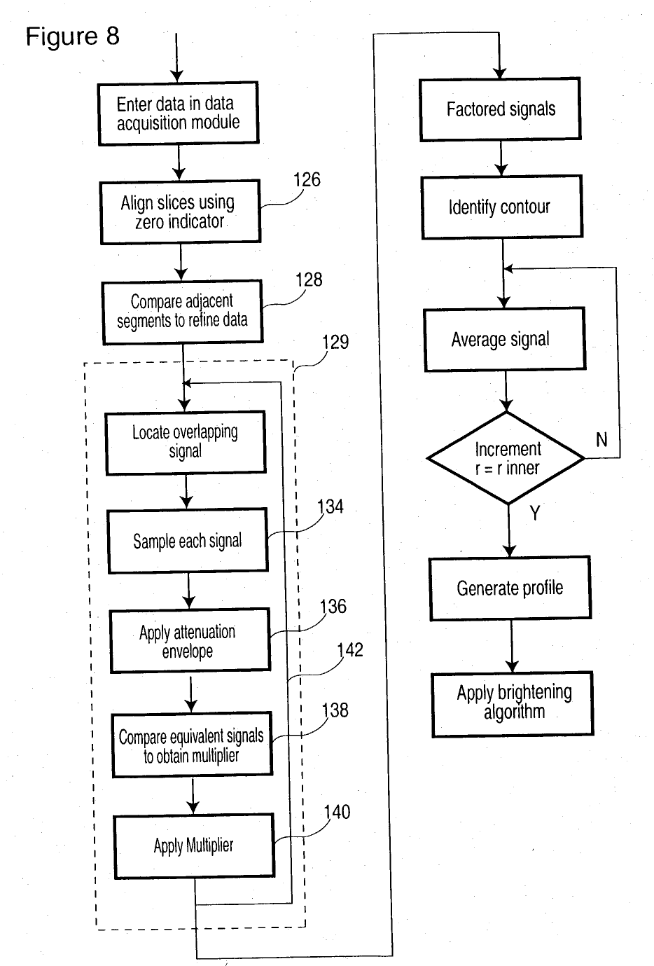

As shown schematically in FIG. 7, after one revolution, the data acquisition module 25 acquires data representing a set of conical slices S₁, S₂, S₃ made up of segments 124 representing the signals obtained from transducers 82, 84, and 86 respectively. Each subsequent scan obtains data representing an additional set of slices displaced vertically from the previous set to provide a set nested conical slices from which a three dimensional image may be generated. The data acquisition module 25 receives data including the digitised reflected signal received by each of the transducers and the circumferential and vertical position of the transducer when the signal was received. Each of the data records, therefore is a representative of the structure along respective segments 124 of the conical slices S₁, S₂, S₃, shown in FIG. 7. To permit generation of a three dimensional image, the data in the data acquisition module 25 must be manipulated as set out in the flow chart of FIG. 8.

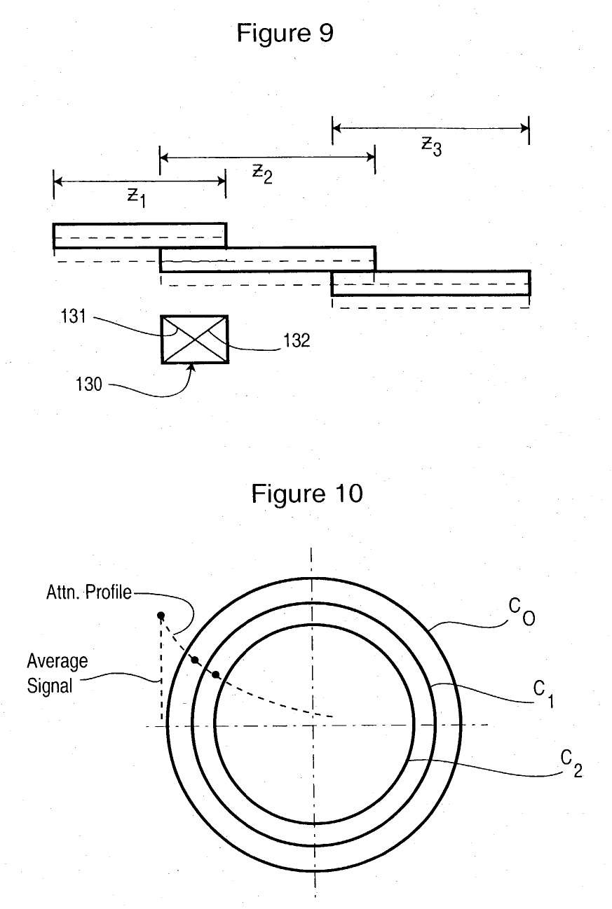

As an initial step 126, the slices S₁, S₂, S₃ are aligned circumferentially using the zero reference signal obtained from the indexing function 107. As indicated in FIG. 9, the segments 124 in one slice will be offset circumferentially from one segment in the adjacent slice due to the rotation of the scanner assembly. The width of the insonifying beam produces a degree of overlap between circumferentially adjacent segments 124 which increases as the segments 124 converge toward the apex of the cone. The circumferentially adjacent segments will therefore contain common information and a comparison of the adjacent segments 124, indicated at 128 in FIG. 8, permits confirmation of a detected structure and removal of noise from the received signal.

The segments 124 in each slice require further processing to take account of the different gains established in preamplifiers 106 for each of the transducers 82, 84, 86. Due to the overlapping between the focal zones the Z₁, Z₂, Z₃ of each transducer, a comparison indicated at 129 between the same signal in the overlapping portions may be used to produce a normalising multiplier to provide a common basis for the signal in each segment.

Referring therefore to FIG. 9, the segments 124 for the zones Z₁ and Z₂ overlap radially as indicated at 130. The signal in each of the overlapping portions of the segments should be representative of the same structure and therefore have the same value. To compensate for signal attenuation, a weighting factor is applied as indicated by the trapezoidal envelope's 131, 132 associated with zones Z₁ and Z₂ respectively. The signal of each segment is sampled (134) at a number of locations in the overlapping portion 130 weighted (136) by the appropriate envelope 131, 132 and a comparison made between the resulting values to obtain (138) a ratio between the signals. The ratio is then applied (140) to one of the segments 124. A similar process is repeated (142) in the overlap between Z₂ and Z₃ so that the three segments representing the structure at a particular location are represented on a common basis.

The factored received signals obtained at step (140) are still influenced by the attenuation of the beam as it passes through the insonified tissue. To compensate for the attenuation in the direction of propagation, an average attenuation profile is obtained from the factored signals as indicated in FIG. 10.

Initially, the contour C₀ of the surface of the breast is located by examining each composite received signal for the initial reflection. The detected contour C₀ is decreased by a predetermined offset, dr, and the average signal about the circumference of the decreased contour C₁ is obtained. The contour is further decreased by the offset dr and the average signal C₂ is obtained. This is repeated along the path of the propagation to an inner value to obtain an attenuation profile indicated in chain dot line based on the average signal. Once the profile is determined, an inverse or reciprocal of the profile is obtained and a brightening algorithm applied to the reformatted signals to obtain a normalised set of data representative of the structure that has been insonified.

After each circumferential scan has been normalised, the data may then be converted to a 3D Cartesian representation for display.

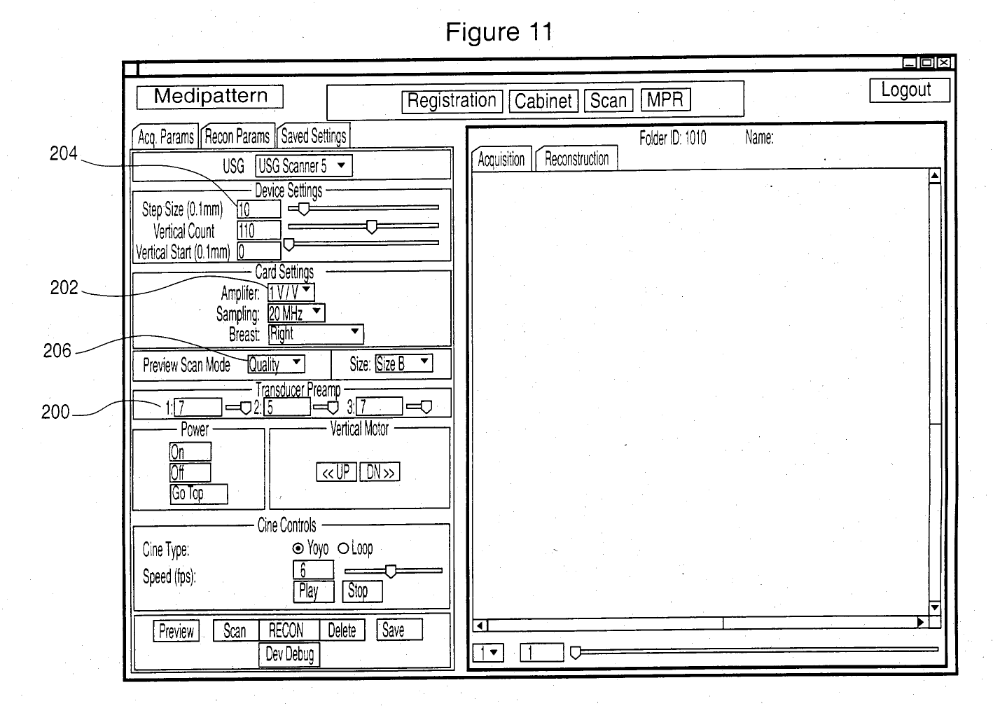

The software application implemented by the computer 24 includes a GUI interface, shown in FIG. 11, permitting selection of the parameters required for the data acquisition and identification of the patient, date and similar biographical data. The parameters selected include gain control for each of the preamplifiers 106 to compensate for signal attenuation on a transducer by transducer basis as indicated at 200; an adjustable delay period between pulse control multiplexer and signal multiplexer to compensate for different tissue sample sizes; adjustable amplification of the main amplifier 104 to compensate for tissue density fluctuations between patients indicated at 202; adjustable vertical increments 204 at the head 56 to accommodate different pathologic conditions and adjustable horizontal scan rate 206 to optimise the data acquisition versus the time required to perform a scan. Each of these parameters is user selectable on the GUI for a particular patient and transmitted to the control and synch module 102 prior to a scan being performed.

To perform a scan the drum 42 is filled with water such that upon placement of the breast within the drum the water will be within a few mm of the top of the drum 42. A patient is positioned on the patient support surface 14 and the scanner support base 26 adjusted so that the table 18 is slightly proud of the support surface 14. The table 18 is positioned such that the majority of the weight of the patient is taken by the support surface 14 but there is sufficient pressure exerted on the table 18 by the patient to maintain a stable position and an acceptable level of comfort. Relative movement between the scanner and patient due to breathing or bodily movement is thus inhibited by the extra pressure exerted by the body. The patient/s breast is allowed to hang pendant within the water.

The patient's arm on the opposite side to the breast being examined may be placed above their head to increase contact, improve comfort and reduce the compression of tissue adjacent the chest cavity. After initial positioning, a visual check is made for correct positioning and a preview or test scan is initiated to confirm a proper position. Once the correct position is obtained, the patient is immobilised by padding and/or straps on the support surface 14.

To facilitate data acquisition and identification, a pair of presence transducers 122, typically either proximity or pressure transducers, are incorporated in the table 18 to either side of the aperture. The presence of the patient's sternum will be detected by one of the transducers 122 thereby indicating whether the left or right breast is being imaged.

The operating parameters are selected at the GUI and a scan initiated. The switch 63 ensures that the start position of head 56 is maintained for each scan. At each designated interval as identified by the encoder 70, the respective one of transducers 82, 84, 86 is pulsed and the reflected image received and processed. The overlapping focal zones Z₁ Z₂ Z₃ ensure a complete scan along the axis of propagation. The delay between propagation and receipt of the signal at each transducer ensures that the acquisition is performed in the region of interest for each transducer in turn and that the initial large reflected signal obtained during initial pulse firing is avoided. As seen in FIG. 5, the signals processed are from distal portions of the path so that the signal attenuation is minimised over the observed depth of field. This permits the gain to be adjusted to obtain optimum signal strength and thereby enhance image acquisition and for each transducer to be adjusted for optimum imaging in its zone.

As can also be seen in FIG. 5, the inclination of the transducers permits insonification of the breast above the portion immersed in water and beyond the drum. This permits interrogation of the chest tissue surrounding the breast, including the lymphatic system, and thereby provides a more comprehensive scan. The inclination of the transducer also brings the angle of incidence between the signal and surface of the breast closer to 90 degrees, to improve signal penetration and reconstruction

The circumferential scan may proceed either incrementally position by position or, preferably, on a continuous basis. The rate of scan may be modulated by adjusting the rotational speed of the drum 42 and the resolution adjusted by sampling at multiples of marks on the encoder 70. When the encoder 70 detects the zero position, a full rotation has been achieved and the transducer head 56 is displaced vertically along the Z-axis and the next scan (slice) begins.

The data acquisition continues until the full vertical scan is complete. The data acquired during scanning from each transducer represents a series of conical slices, which are reconstructed as described above to obtain a three-dimensional ultrasound image of the breast is thus obtained.

The image quality is enhanced by utilising distal portions of each of the ultrasound signals and by pre-amplifying the received signals on the electronic circuitry 80 carried by the drum and placed in immediate proximity to transducers. This amplification of signal reduces the effect of noise inherent in transmission across the slip ring to result in an increased signal to noise ratio and preserve.

The scanning system is relatively simple in construction and may be used without specialised installation, supervision and safety measures. The patient support facilitates repeatable image scans. Thus, although a single image may be obtained, the scanner assembly may also be used to obtain images on a periodic basis under comparable imaging conditions. The periodic images can then be compared and changes in detected structure noted. The computer 26 may be remote from the scanning apparatus so that data acquisition may be performed over a telephone connection or similar communication line. If preferred, a communication module may be incorporated into the base allowing the scanner to function as a client in a LAN or other network. In this manner the comparison of images may be performed remotely and the image parameters adjusted remotely to enhance data acquisition. The provision of the network capability also permits multiple scanners to be controlled from a single operator workstation and for data to be acquired and processed centrally.

The system is designed for non-real time analysis, remote analysis and time series analysis. The support system permits the patient to be scanned in a non-deformed “configuration”, where the breast is placed in such a way as to allow for a reasonable ability to repeat the breast position from scan to scan over time. This allows for the scans to be mapped to each other and enables the diagnostician to compare scans and determine whether any changes are occurring in the tissue. The apparatus facilitates the acquisition of such scans in a home or clinic environment with remote monitoring and diagnosis. If further investigation is warranted it may be performed on the same apparatus under different operating parameters, e.g. resolution, or as an alternative form of scan at a specialised facility.

Although, the system has been described with a breast-imaging scanner, it will be appreciated that it may be adapted for other soft tissue imaging with appropriate modification to the drum and pertinent support.

The embodiments of the invention in which an exclusive property or privilege is claimed are defined as follows:

1. A medical imaging system comprising a patient support surface, an imaging apparatus having a support table located within said support surface, and adjustable relative to said support surface to be located above said surface and thereby engage a portion of said patient to be imaged.

2. A medical imaging system according to claim 1 wherein said imaging apparatus includes a chamber to receive a portion of said patient to be imaged, said table having an aperture to permit positioning of said portion in said chamber.

3. A medical imaging system according to claim 2 wherein a sensor is provided on said table to indicate orientation of said patient.

4. A medical imaging system according to claim 3 wherein a pair of sensors is provided at diametrically opposed locations for detection of body orientation.

5. An ultrasound scanner assembly comprising a base, a drum rotating on said base, a transducer head rotating with said drum and displaced relative to said drum along an axis parallel to the axis of rotation, said head including a plurality of transducers each operable to propagate a wave along an axis of propagation and to receive signals from respective focal zones spaced relative to one another along said axis of propagation.

6. A scanner assembly according to claim 5 wherein said axis of propagation is inclined to said axis of rotation.

7. A scanner assembly according to claim 6 wherein said axis of propagation is inclined at an angle generally orthogonal to the average target surface of a portion of a patient within said drum.

8. A scanner assembly according to claim 6 wherein said angle of propagation is between 65° and 55° to said axis of rotation.

9. A scanner assembly according to claim 8 wherein said angle of propagation is 60° to said axis of rotation.

10. A scanner assembly according to claim 5 wherein said zones are located at a portion of said wave remote from the transducer and said angle of propagation permits insonification of a region beyond said drum.

11. A scanner assembly according to claim 7 wherein said transducers are interfaced to and controlled by an electronic circuit mounted on said drum for rotation therewith.

12. A scanner assembly according to claim 11 wherein said electronic circuit includes an amplifier to adjust the gain of signals received by respective ones of said transducer.

13. A scanner assembly according to claim 7 wherein said transducers are located in a channel in fluid communication with said drum.

14. A scanner assembly according to claim 7 including an encoder to indicate the position of said drum about the axis and thereby control operation of said transducers at predetermined intervals.

15. A scanner assembly according to claim 16 wherein said encoder includes a registration position and movement of said transducer head relative to said drum is initiated upon attaining said registration position.

16. A scanner assembly according to claim 17 including a signal indicative of a limit of movement of said transducer head relative to said drum.

17. A scanner assembly according to claim 5 including a data transfer interface to transfer data over a network to a recipient.

18. A scanner assembly according to claim 5 wherein said focal zones overlap and data from each signal in overlapping portions of said focal zones is compared to conform said signals to a common base.

19. A scanner assembly according to claim 18 wherein each of said signals is adjusted for attenuation in said overlapping portions prior to comparison.

20. A scanner assembly according to claim 18 wherein an attenuation profile is obtained from an evaluation of data collected during a scan to application to said signals.

21. A method of monitoring a medical condition through insonification with ultrasound by locating a portion of a patient on a scanner in a predetermined position to permit acquisition of date in a repeatable manner, conducting a succession of scans of the area of interest of the patient at predetermined intervals, transferring the scan to a remote location and comparing time separated scans to determine changes in said medical condition.
